(19) United States
(12) Patent Application Publication
(10) Pub. No.: US 2003/0065231 A1
(43) Pub. Date: Apr. 3, 2003

(54) Methods for preparing primary alkyl bromides

(76) Inventors: David T. Erdman, Liberty, MO (US); Jonathan D. Spicher, Parkville, MO (US); Mary F. Lamar, Overland Park, KS (US)

Correspondence Address: BAYER CORPORATION, PATENT DEPARTMENT, 100 BAYER ROAD, PITTSBURGH, PA, 15205, US

(21) Appl. No.: 09/970,841

(22) Filed: Oct. 3, 2001

(51) Int. Cl. C07C 17/093

(52) U.S. Cl. 570/235

(57) ABSTRACT

Methods of converting methyl chloride to methyl bromide include the steps of providing a composition comprising a bromide salt and a liquid; contacting the composition with gaseous methyl chloride and/or methyl chloride dissolved in a water-immiscible solvent; and recovering methyl bromide.

### FIELD OF THE INVENTION

This invention relates to methods of preparing primary alkyl bromides, particularly methyl bromide. The invention also relates to methods of converting methyl chloride to methyl bromide and to methods of reducing the level of bromide salt in aqueous compositions.

### BACKGROUND OF THE INVENTION

Primary alkyl bromides, such as methyl bromide, may be used as fumigants, alkylating agents, or intermediates in the synthesis of other chemical compounds, in particular pharmaceutical or agricultural chemicals.

Methyl bromide may be prepared by treating methanol with hydrobromic acid and sulfuric acid. Unfortunately, such processes required the handling and/or disposing noxious and/or dangerous compounds.

Salt exchange reactions include reactions wherein an alkyl halide is converted to another alkyl halide, such as, for example, the Finkelstein reaction wherein alkyl chlorides or bromides are converted to alkyl iodides using solid potassium or sodium iodide in acetone. Unfortunately, such reactions require the handling or large volumes of acetone, and the equilibrium constants for alkyl chloride/alkyl bromide exchanges in acetone often favor the alkyl chloride.

Hughes et al., J. Chem. Soc., 3173-3175 (1955), teach that the displacement of bromine from alkyl bromides using lithium chloride in dry acetone is only mildly reversible. Starks and Liotta, Phase Transfer Catalysis”, Academic Press, New York, N.Y. (1978), teach the use of phase transfer catalysts in halide exchange reactions in two-phase systems and in homogenous organic reactions, and disclose that the displacement of chloride ion on octyl bromide is faster than bromide ion displacement on octyl chloride.

Handling large volumes of acids may be difficult and/or costly. Additionally, processes using large amounts of sulfuric acid may generate an aqueous waste stream comprising NaSO₄ as well as residual sulfuric acid, and NaSO₄ generally causes more environmental problems than sodium salts such as NaCl or NaBr. Thus, there is a need for facile methods of producing primary alkyl bromides which do not require the use of large volumes of acids.

As handling large volumes of some organic solvents may be difficult and/or costly, there is a need for methods of producing primary alkyl bromides which do not require the use of large volumes of solvents such as acetone.

There is also a need for methods of reducing the level of bromide salts in aqueous compositions.

There is a need for methods of regenerating alkyl bromides from bromide salts which are formed when alkyl bromides, such as methyl bromide, are used as reactants.

### SUMMARY OF THE INVENTION

Accordingly, it is an object of the present invention to obviate problems of the prior art.

It is a further object of the present invention to provide methods of preparing alkyl bromides.

It is also an object of the present invention to provide methods of preparing alkyl bromides which do not require the handling, storage or disposal of large volumes of acetone, hydrobromic acid or sulfuric acid.

It is another object of the present invention to provide methods of removing bromide salts from liquid compositions.

It is a further object of the present invention to provide methods of regenerating alkyl bromides from bromide salts.

These and additional objects are provided by the methods of the invention.

In one embodiment the invention is directed to methods of converting methyl chloride to methyl bromide comprising:

(a) providing a composition comprising bromide salt and a first organic solvent;

(b) contacting the composition with gaseous methyl chloride and/or methyl chloride dissolved in a second organic solvent; and

(c) recovering a gaseous product comprising methyl bromide.

The first and second organic solvents may be the same or different.

In another embodiment the invention is directed to methods of preparing a primary alkyl bromide comprising:

(a) providing a composition comprising bromide salt and an organic solvent;

(b) contacting the composition with a primary alkyl chloride thereby forming a primary alkyl bromide; and

(c) recovering the primary alkyl bromide.

In a further embodiment the invention is directed to methods of converting methyl chloride to methyl bromide comprising:

(a) providing a column oriented substantially vertically along its axis and having an upper portion and a lower portion, wherein the column contains packing suitable for continuous extractions;

(b) introducing an organic feed into the upper portion of the column, wherein the organic feed comprises a water immiscible organic solvent and a chloride salt selected from the group consisting of quaternary ammonium salts, quaternary phosphonium salts, alkali metal chlorides, alkaline earth metal chlorides and combinations thereof, and/or a catalyst selected from the group consisting of crown ethers, cryptanes, PEGs, TDA-1, and combinations thereof;

(c) introducing an aqueous solution of bromide salt into the lower portion of the column;

(d) allowing at least one portion of the organic feed and at least a portion of the aqueous solution of bromide salts feed to contact each other within the column thereby obtaining an organic phase comprising bromide salt; and

(e) treating the organic phase comprising bromide salt with methyl chloride to generate methyl bromide and to regenerate the chloride salt and/or catalyst.

The step of treating the organic phase comprising bromide salt with methyl chloride may occur in a separate reactor, that is, a reactor other than the column used to prepare the organic phase.

In yet another embodiment the invention is directed to methods of reducing the level of bromide salt in an aqueous composition comprising bromide salt and water, the method comprising the step of extracting the aqueous composition with an organic composition comprising an organic solvent and a phase transfer catalyst to obtain an organic phase comprising the organic solvent, phase transfer catalyst and bromide salt. The methods may further comprise the step of heating the organic phase in the presence of an primary alkyl chloride.

These and additional aspects, objects and advantages of the invention are more fully described in the following detailed description.

### DETAILED DESCRIPTION

The present invention is directed to the preparation of primary alkyl bromides in the presence of a liquid. The present invention is also directed to methods of reducing the level of bromide salts in an aqueous solution, and methods of regenerating alkyl bromides.

Methods in accordance with the invention avoid the use of large volumes of acids and/or organic solvents such as acetone. Thus any resulting aqueous waste stream may be easily handled and/or treated.

The primary alkyl bromides are prepared by providing a composition comprising bromide salt and a liquid; contacting the composition with a primary alkyl chloride thereby forming a primary alkyl bromide; and recovering the primary alkyl bromide. Methods in accordance with the invention may be used to regenerate alkyl bromides from bromide salts which are formed when alkyl bromides are used as reactants.

While not being bound by theory, it is believed that the reaction proceeds as set forth below:

R−X+ZBr→R−Br+ZX

wherein R is a primary alkyl, preferably a C₁-C₂₀ primary alkyl, more preferably a C₁-C₅ alkyl; even more preferably a C₁-C₂ alkyl; X is a halogen, preferably a halogen other than Br, more preferably Cl; Z is a cation such hydrogen, sodium, potassium, lithium, magnesium, calcium, copper which is coordinated by a organic soluble catalyst, or a quaternary ammonium or phosphonium cation, preferably Z is quaternary ammonium or phosphonium hydrogen, sodium, potassium or lithium.

In one embodiment of the invention an aqueous composition comprising bromide salt is extracted with an organic solvent in the presence of a phase transfer catalyst. The resulting organic phase comprising organic solvent, bromide salt and phase transfer catalyst is then contacted with gaseous primary alkyl chloride and/or primary alkyl chloride dissolved in a water-immiscible solvent. Primary alkyl bromides are produced. While not being bound by theory, it is believed that during the extraction of the aqueous composition with the organic solvent the bromide ion exchanges with the anion of the phase transfer catalyst, and during contact with the primary alkyl chloride the bromide of the quaternary compound exchanges with the chloride.

In one embodiment of the invention an aqueous composition comprising water and a bromide salt of the formula YBr, wherein Y is a cation such hydrogen, sodium, potassium, lithium, magnesium, calcium, copper or copper which is coordinated by a organic soluble catalyst, and an organic composition comprising an organic liquid and a salt of the formula ZX, wherein X is a halogen, preferably a halogen other than Br, more preferably Cl and Z is a cation such hydrogen, sodium, potassium, lithium, magnesium, calcium, copper which is coordinated by a organic soluble catalyst, or a quaternary ammonium or phosphonium cation, preferably a quaternary ammonium or phosphonium, hydrogen, sodium, potassium or lithium, are provided. The aqueous composition and the organic composition are mixed, resulting in the formation of an organic phase comprising organic solvent and a bromide salt. While not being bound by theory, this step, referred to as the extraction step, is believed to proceed as set forth below:

ZX_{(organic)}+YBr_{(aqueous)}→ZBr_{(organic)}+YX_{(aqueous)}

As used herein, “extraction” refers to the removal of a least a portion of the bromide ion form the aqueous composition into the organic composition. Z and Y may be the same or different.

The organic phase comprising the organic solvent and the bromide salt is then treated with a primary alkyl of the formula R-X, wherein R is a primary alkyl, preferably a C₁-C₂₀ primary alkyl, more preferably a C₁-C₅ alkyl; even more preferably a C₁-C₂ alkyl; X is a halogen, preferably a halogen other than Br, more preferably Cl. While not being bound by theory, it is believed that the reaction proceeds as set forth below:

R−X+ZZBr→R−Br+ZX

Suitable aqueous phase bromide salts (YBr) include hydrogen bromide, alkali metal bromides, alkaline earth metal bromides, organic quaternary Group V bromides and combinations thereof. In one embodiment the bromide salt is selected from the group consisting of sodium bromide, potassium bromide, lithium bromide, hydrogen bromide and combinations thereof, while in another embodiment the bromide salt is selected from the group consisting of organic quaternary bromides and mixtures thereof.

The aqueous phase bromide salt (YBr) may be present in a mixture with salts other than bromide salts, such as sodium sulfate and sodium chloride. The bromide salt may be present in the aqueous composition at a concentration of from about 1% to about 50%, preferably from about 5% to about 35%, by weight.

Suitable organic phase bromide salts (ZBr) may be selected from the group consisting of organic quaternary nitrogen bromide, organic quaternary phosphorous bromide, organic quaternary arsenic bromide, organic quaternary antimony bromide, organic quaternary bismuth bromide and combinations thereof, preferably the organic quaternary bromide selected from the group consisting of organic quaternary nitrogen bromide, organic quaternary phosphorous bromide, and combinations thereof. The organic groups of the organic quaternary bromide are each independently selected from benzyl or monovalent hydrocarbon radicals having from 1 to 20 carbon atoms. Suitable organic quaternary bromides include tri(octyl)methylammonium bromide, tetra(octyl)ammonium bromide, benzyl tributyl ammonium bromide, tetrabutylammonium bromide, tetrabutyl phosphonium bromide and combinations thereof.

The aqueous phase bromide salt (ZBr) may be extracted from the aqueous composition into the water-immiscible organic solvent using a phase transfer catalyst. The phase transfer catalyst is present in an amount sufficient to aid the extraction of the bromide from the aqueous phase to the organic phase. In one embodiment the bromide salt is selected from alkali metal bromides, alkaline earth metal bromides and combinations thereof, and is extracted in the presence of a phase transfer catalyst.

Suitable phase transfer catalysts may be selected from the group consisting of crown ethers, cryptanes, polyalkylene glycols such as poly(C₂-C₄ alkylene glycols), tris[2-(2-methoxyethoxy)-ethyl]amine (TDA-1) and combinations thereof. Amines or phosphines which are quaternized under the reaction conditions may also be used as phase transfer catalysts.

In one embodiment the phase transfer catalyst is an organic quaternary Group V salt selected from the group consisting of organic quaternary nitrogen salts, organic quaternary phosphorous salts, organic quaternary arsenic salts, organic quaternary antimony salts, organic quaternary bismuth salts, and combinations thereof. The organic groups of the organic quaternary Group V salt may selected from benzyl and monovalent hydrocarbon radicals, preferably from benzyl or monovalent hydrocarbon radicals having from 1 to 20 carbon atoms.

In one embodiment of the invention the catalyst is a quaternary ammonium or phosphonium salt of the structure:

R¹R²R³R⁴Q⁺X⁻

wherein R¹, R2, R3 and R⁴ are each independently selected from benzyl and alkyl, preferably from benzyl and monovalent hydrocarbon radicals having from 1 to 20 carbon atoms; Q is nitrogen or phosphorus; and Z is an anion such as bromide, chloride, sulfate or hydroxy. Suitable organic quaternary salts include tri(octyl)methylammonium salts, tetra(octyl)ammonium salts, benzyl tributyl ammonium salts, tetrabutylammonium salts, tetrabutyl phosphonium salts and combinations thereof.

The phase transfer catalyst may also be present during the conversion of the primary alkyl chloride to the primary alkyl bromide in an amount sufficient to catalyze the conversion. For example the phase transfer catalyst may be present in an amount of from about 0.5 to about 50, preferably from about 1 to about 25, mole percent based on total moles of added primary alkyl chloride. In one embodiment of the invention the phase transfer catalyst is an organic quaternary Group V salt and the primary alkyl chloride is methyl chloride, and the organic quaternary Group V salt is present in an amount of from about 0.5 to about 50 mole percent based on total moles of methyl chloride added.

The reaction liquid comprises as an organic solvent. As used herein, “reaction liquid” refers to the liquid present during the treatment of the bromide salt with the alkyl chloride. Preferred organic solvents include water-immiscible organic solvents. The water-immiscible organic solvent may be selected from the group consisting of C₅-C₂₀ aliphatic and cycloaliphatic hydrocarbon solvents, mono- and polycyclic aromatic solvents, di(C₁-C₄ alkyl)form-amides, ketones such as C₄-C₈ ketones, and combinations, thereof. The amount of organic solvent, preferably water-immiscible organic solvent, in the reaction liquid may be a weight of organic solvent of from about 2 times to about 50 times, preferably from about 5 times to about 20 times, the weight of the primary alkyl chloride.

The preparation of the primary alkyl bromide preferably occurs in the substantial absence of water. As used herein, “The substantial absence of water” is intended to mean that the total water comprises no more than 10%, preferably less than about 5%, by weight of the reaction mixture comprising the bromide, any catalyst, liquid and primary alkyl chloride. Preferably the liquid is a water-immiscible organic solvent and the primary alkyl chloride is methyl chloride. In one embodiment the preparation of the primary alkyl bromide occurs in the substantial absence, preferably the absence, of acetone.

As used herein “alkyl” refers to both linear and branched chain alkyls. As used herein “C₅-C₂₀ aliphatic and cycloaliphatic hydrocarbon solvents” refer to hydrocarbons having from 5 to 20 carbons atoms in the hydrocarbon backbone and which may be substituted or unsubstituted. Suitable substituents include halides, ethers, esters, cyano, amides, alkenes, alkynes and combinations thereof.

As used herein “mono- and polycyclic aromatic solvents” refer to solvents containing one or more cyclic moiety. The cyclic moiety may contain a heteroatom in the ring, and may be substituted or unsubstituted. As used herein, “heteroatom” refers to atoms other than carbon, such as nitrogen, oxygen and sulfur. Suitable substituents include C₁-C₉ alkyl groups, nitro, halides, cyano, esters, ethers, amides and combinations thereof. Suitable mono- and polycyclic aromatic solvents include benzene, toluene, pyridine, furan, thiophene, piperidine.

Typical water-immiscible organic solvents include benzene, toluene, xylene, methyl cyclohexane, methyl iso-butylketone, ethyl acetate, methyl-tert-butyl ether, chloroform, dichloromethane, ethyl ether, hexane, heptane, octanol and combinations thereof. Preferred water immiscible organic solvents include toluene and methyl iso-butylketone.

As used here, “primary alkyl chloride” is intended to refer to compounds having a chloride on a terminal alkyl carbon, and is intended to include arylalkyls having a chloride on a terminal alkyl carbon. The primary alkyl chloride molecules generally have from about 1 to about 20, preferably from about 1 to about 5, more preferably from about 1 to about 2, carbon atoms. Suitable primary alkyl chlorides include 1-chlorobutane, 1-chloroethane, methyl chloride, benzyl chloride, or mixtures thereof. Preferably the primary alkyl chloride is methyl chloride.

The bromide salt is present in an amount to provide a molar equivalent ratio of bromide ions to primary alkyl chloride of from about 1:10 to about 10:1. In one preferred embodiment the primary alkyl chloride is methyl chloride, and the bromide salt is present in an amount to provide a molar equivalent ratio of bromide ions to methyl chloride of from about 1:10 to about 10:1

The methods in accordance with the present invention may be performed at any pressure and temperature sufficient for the desired reaction to occur. Generally the methods are performed at a temperature of from about 40° C. to about 200° C., preferably from about 60° C. to about 80° C., and at pressure of from about 0 to about 20 atmospheres.

The conversion of the primary alkyl chloride to primary alkyl bromide may be performed batchwise, semi-batchwise or continuously, and gases produced during the conversion may be purified by continuous or batchwise distillation, or by pressure swing absorption methods.

Any suitable reactor may be used, including fixed-bed reactors having an immobilized phase transfer catalyst. For example, one embodiment of the invention comprises:

(a) providing a column oriented substantially vertically along its axis and having an upper portion and a lower portion, wherein the column contains packing suitable for continuous extractions;

(b) introducing an organic feed into the upper portion of the reaction column, wherein the organic feed comprises an organic solvent; a bromide salt, preferably a bromide salt selected from the group consisting of the alkali metal bromides, alkaline earth metal bromides and combinations thereof; and, optionally, a phase transfer catalyst;

(c) introducing methyl chloride gas into the lower portion of the reaction column; and

(d) allowing at least one portion of the organic feed and at least a portion of the methyl chloride to contact each other within the column thereby converting at least a portion of the methyl chloride to methyl bromide.

Another embodiment of the invention comprises an extraction and a reaction. The extraction comprises the steps of:

(a) providing a column oriented substantially vertically along its axis and having an upper portion and a lower portion, wherein the column contains packing suitable for continuous extractions;

(b) introducing an aqueous feed into the upper portion of the column, wherein the aqueous feed comprises water and a bromide salt selected from the group consisting of the alkali metal bromides, alkaline earth metal bromides and combinations thereof;

(c) introducing an organic feed into the lower portion of the column, wherein the organic feed comprises a water immiscible organic solvent, and a quaternary ammonium salt, quaternary phosphonium salt, or a coordinating catalyst such as crown ether, cryptane, PEGs, TDA-1 and combinations thereof; and

(d) allowing at least one portion of the aqueous feed and at least a portion of the organic feed to contact each other within the column thereby extracting at least a portion of the bromide salts into the organic phase.

The reaction comprises the steps of:

(a) providing a reaction column oriented substantially vertically along its axis and having an upper portion and a lower portion, wherein the reaction column contains packing suitable for continuous extractions;

(b) introducing an organic feed into the upper portion of the reaction column, wherein the organic feed comprises a water immiscible organic solvent and a bromide salt selected from the group consisting of quaternary ammonium salts, quaternary phosphonium salts, alkali metal bromides, alkaline earth metal bromides and combinations thereof, and a catalyst selected from the group consisting of crown ethers, cryptanes, PEGs, TDA-1, and combinations thereof;

(c) introducing methyl chloride gas into the lower portion of the reaction column; and

(d) allowing at least one portion of the organic feed and at least a portion of the methyl chloride feed to contact each other within the reaction column thereby converting at least a portion of the methyl chloride to methyl bromide.

Methods in accordance with the present invention provide good recoveries of the bromide salt. Generally at least about 30%, preferably at least about 50%, more preferably at least about 60%, by weight, of the bromide salt is converted to primary alkyl bromide. In one embodiment, the primary alkyl bromide is methyl bromide, and at least about 50%, preferably at least about 75%, more preferably at least about 80%, even more preferably at least about 90%, of the bromide salt is converted to methyl bromide. In one embodiment of the invention any unreacted primary alkyl chloride is recovered and may be used recycled in a subsequent bromide salt conversion.

Excessive hydrolysis of the primary alkyl bromide product can be avoided using methods of the present invention. Generally no more than about 25%, preferably no more than about 10%, more preferably no more than about 5%, by weight, of the primary alkyl bromide undergoes hydrolysis.

Methods in accordance with the present invention may be used to reduce the level of bromide salt in aqueous compositions. Accordingly, the method may comprise converting at least a portion of a bromide salt in an aqueous composition to primary alkyl bromides, and removing the primary alkyl bromides from the aqueous composition. The primary alkyl chloride may be selected from the group consisting of 1-chlorobutane, 1-chloroethane, methyl chloride, benzyl chloride, and combinations thereof, preferably the primary alkyl chloride in methyl chloride. The bromide salt may be selected from the group consisting of alkali metal bromides, alkaline earth metal bromides, organic quaternary bromides and combinations thereof. A phase transfer catalyst, preferably a phase transfer catalyst selected from the group consisting of organic quaternary Group V salts, crown ethers, polyalkylene glycols such as poly(C₂-C₄ alkylene glycols), tris[2-(2-methoxyethoxy)-ethyl]amine and combinations thereof, may be added to the composition at a level sufficient to facilitate phase transfer.

The bromide salt may be present in the aqueous composition at a level of from about 1% to about 50%, by weight of the composition, preferably at a level of from about 5% to 35%, by weight of the composition. The process may be performed at a temperature of at least about 40° C., preferably from about 40° C. to about 200° C., more preferably from about 50° C. to about 90° C., and at a pressure of from about 0 to about 20 atmospheres.

Generally at least about 30%, preferably at least about 50%, more preferably at least about 60%, by weight, of the bromide salt is removed from the aqueous compositions.

In one embodiment of the invention a method of reducing the level of bromide salt in an aqueous composition comprising bromide salt and water, the method comprising the step of extracting the aqueous composition with an organic composition comprising an organic solvent and a phase transfer catalyst to obtain an organic phase comprising the organic solvent, phase transfer catalyst and bromide salt. The primary alkyl chloride may be methyl chloride, and the phase transfer catalyst is selected from the group consisting of organic quaternary Group V salts, crown ethers, polyalkylene glycols such as polyethylene glycols, cryptanes, tris[2-(2-methoxyethoxy)-ethyl]amine and combinations thereof. The method may further comprise the step of heating the organic phase in the presence of an primary alkyl chloride. The organic phase may to a temperature of at least about 40° C., preferably of from about 40° C. to about 200° C.

Throughout the examples and the present specification, parts and percentages are by weight unless otherwise specified. The following example is illustrative only and is not intended to limit the scope of the methods of the invention as defined by the claims.

### EXAMPLES

### Example 1

About 2250 g toluene and about 250 g trioctylmethylammonium chloride are mixed and the resulting organic solution is extracted three times with a saturated aqueous sodium bromide solution. The organic solution is placed in an autoclave and is heated to a temperature in the range of from about 71° C. to about 75° C. The system is evacuated and methyl chloride gas is introduced. The pressure of the vessel is set at 45 psi. At equilibrium the gas phase comprises about 6%, by weight, methyl bromide. About one third of the bromide salts are consumed. Extraction of the reaction mixture with water and measurement of pH indicates that there is little hydrolysis of the resulting methyl bromide.

### Example 2

About 2250 g toluene and 250 g trioctylmethylammonium chloride are mixed and the resulting organic solution is extracted three times with a saturated aqueous sodium bromide solution. The organic solution is placed in an autoclave and is heated to a temperature in the range of from about 41° C. to about 55° C. The system is evacuated and methyl chloride gas is introduced. The pressure of the vessel is set at 20 psi. At equilibrium the gas phase comprises about 6%, by weight, methyl bromide. About one third of the bromide salts are consumed. Extraction of the reaction mixture with water and measurement of pH indicates that there is little hydrolysis.

Additional embodiments and modifications within the scope of the claimed invention will be apparent to one of ordinary skill in the art. Accordingly, the scope of the present invention shall be considered in terms of the following claims, and is understood not to be limited to the details of the methods described in the specification.

What is claimed is:

1. A method of converting methyl chloride to methyl bromide comprising:
(a) providing a composition comprising bromide salt and a first organic solvent;
(b) contacting the composition with gaseous methyl chloride and/or methyl chloride dissolved in a second organic solvent; and
(c) recovering a gaseous product comprising methyl bromide;
wherein the first and second organic solvents may be the same or different.

2. A method according to claim 1, wherein the step of providing the composition comprising bromide salt and first organic solvent comprises the step of extracting an aqueous solution comprising water and bromide salts with a non-aqueous solution comprising an organic solvent and a phase transfer catalyst, thereby obtaining the composition comprising bromide salt and a first organic solvent

3. A method according to claim 1, wherein the bromide salt is an organic quaternary bromide salt.

4. A method according to claim 3, wherein the organic quaternary bromide is soluble in the first organic solvent.

5. A method according to claim 1, wherein the first and second organic solvents are the same or different water-immiscible solvent.

6. A method according to claim 1, wherein the composition comprise an amount of bromide salt sufficient to provide a molar equivalents ratio of bromide ions to methyl chloride is at least about 1:1.

7. A method according to claim 1, wherein the bromide salt is an organic quaternary bromide, and the step of contacting the liquid composition with methyl chloride occurs in the substantial absence of water, alkali metal bromides and alkaline earth metal bromides.

8. A method according to claim 1, wherein the composition comprises a water-immiscible organic solvent and a bromide salt selected from the group consisting of organic quaternary bromides and combinations thereof.

9. A method of preparing a primary alkyl bromide comprising:
(a) providing a composition comprising bromide salt and an organic solvent;
(b) contacting the composition with a primary alkyl chloride thereby forming a primary alkyl bromide; and
(c) recovering the primary alkyl bromide.

10. A method according to claim 9, wherein the primary alkyl chloride is selected from the group consisting of 1-chlorobutane, 1-chloroethane, methyl chloride, benzyl chloride, and combinations thereof.

11. A method according to claim 9, comprising the steps of:
(a) extracting an aqueous composition comprising bromide salt with an organic solvent in the presence of a phase transfer catalyst to obtain an organic phase comprising organic solvent and bromide salt;
(b) contacting the organic phase with a primary alkyl chloride thereby converting at least a portion of the primary alkyl chloride to a primary alkyl bromide; and
(c) recovering the primary alkyl bromide.

12. A method according to claim 9, comprising the steps of:
(a) extracting an aqueous composition comprising bromide salt selected from the group consisting of alkali bromides, alkaline earth metal bromides and combinations thereof with a water-immiscible organic solvent in the presence of an organic quaternary salt to obtain an organic phase comprising the water-immiscible organic solvent and an organic quaternary bromide salt;
(b) contacting the organic phase with a primary alkyl chloride thereby converting at least a portion of the primary alkyl chloride to a primary alkyl bromide; and
(c) recovering the primary alkyl bromide.

13. A method of converting methyl chloride to methyl bromide comprising:
(a) providing a reaction column oriented substantially vertically along its axis and having an upper portion and a lower portion, wherein the reaction column contains packing suitable for continuous extractions;
(b) introducing an organic feed into the upper portion of the reaction column, wherein the organic feed comprises a water immiscible organic solvent and a bromide salt selected from the group consisting of quaternary ammonium salts, quaternary phosphonium salts, alkali metal bromides, alkaline earth metal bromides and combinations thereof, and a catalyst selected from the group consisting of crown ethers, cryptanes, PEGs, TDA-1, and combinations thereof;
(c) introducing methyl chloride gas into the lower portion of the reaction column; and
(d) allowing at least one portion of the organic feed and at least a portion of the methyl chloride feed to contact each other within the reaction column thereby converting at least a portion of the methyl chloride to methyl bromide.

14. A method of reducing the level of bromide salt in an aqueous composition comprising bromide salt and water, the method comprising the step of extracting the aqueous composition with an organic composition comprising an organic solvent and a phase transfer catalyst to obtain an organic phase comprising the organic solvent, phase transfer catalyst and bromide salt.

15. A method according to claim 14, further comprising the step of heating the organic phase in the presence of a primary alkyl chloride.

16. A method according to claim 15, wherein the step of heating the organic phase comprises heating the organic phase to a temperature of at least about 40° C.

17. A method according to claim 15, wherein the step of heating the organic phase comprises heating the organic phase to a temperature of from about 40° C. to about 200° C.

18. A method according to claim 15, wherein the primary alkyl chloride is methyl chloride.

19. A method according to claim 14, wherein the phase catalyst is selected from the group consisting of organic quaternary Group V salts, crown ethers, polyalkylene glycols, tris[2-(2-methoxyethoxy)-ethyl]amine and combinations thereof.

20. A method according to claim 14, wherein the phase catalyst is selected from the group consisting of organic quaternary Group V salts, and combinations thereof.

21. A method of converting methyl chloride to methyl bromide comprises the steps of:
(a) providing a column oriented substantially vertically along its axis and having an upper portion and a lower portion, wherein the column contains packing suitable for continuous extractions;
(b) introducing an organic feed into the upper portion of the column, wherein the organic feed comprises a water immiscible organic solvent and an ingredient selected from a chloride salts; catalysts and combinations thereof;
(c) introducing an aqueous solution of bromide salt into the lower portion of the column;
(d) allowing at least one portion of the organic feed and at least a portion of the aqueous solution of bromide salts feed to contact each other within the column thereby obtaining an organic phase comprising bromide salt; and
(e) treating the organic phase comprising bromide salt with methyl chloride to generate methyl bromide and to regenerate the chloride salt and/or catalyst.

22. A method according to claim 21, wherein the organic feed comprises a chloride salt selected from the group consisting of quaternary ammonium salts, quaternary phosphonium salts, alkali metal chlorides, alkaline earth metal chlorides and combinations thereof.

23. A method according to claim 21, wherein the organic feed comprises a catalyst selected from the group consisting of crown ethers, cryptanes, PEGs, TDA-1, and combinations thereof.
